SEQUENCE LISTING (5 sequences)

SEQ ID NO: 1 - protein, 10 aa, Artificial
  Phage display library peptide
CVKWRGVVVC

SEQ ID NO: 2 - protein, 10 aa, Artificial
  Phage display library peptide
CWSFRNRVLC

SEQ ID NO: 3 - protein, 16 aa, Homo sapiens [sequence omitted]

SEQ ID NO: 4 - protein, 16 aa, Homo sapiens
CATLSWVLQNRVLSSC

SEQ ID NO: 5 - protein, 17 aa, Homo sapiens
CLENFSKWRGSVLSRRC

(19) United States
(12) Patent Application Publication
(10) Pub. No.: US 2010/0310579 A1
(43) Pub. Date: Dec. 9, 2010

(54) METHOD FOR INHIBITING ANGIOGENESIS OR FOR TREATMENT OF CANCER

(75) Inventors: Marko Salmi, Turku (FI); Sirpa Jalkanen, Piispanristi (FI)

Correspondence Address: ROTHWELL, FIGG, ERNST & MANBECK, P.C., 1425 K STREET, N.W., SUITE 800, WASHINGTON, DC, 20005, US

(73) Assignee: Faron Ventures Oy, Turku (FI)

(21) Appl. No.: 12/740,566

(22) PCT Filed: Oct. 15, 2008
(86) PCT No.: PCT/FI2008/050577

§ 371 Date: Apr. 29, 2010

(30) Foreign Application Priority Data
Oct. 30, 2007 (FI) 20070820

(51) Int. Cl. A61K 39/395; C07K 16/18; A61P 35/00; A61P 35/02; C07K 2/00; A61K 38/02; C07H 21/04; A61K 31/713

(52) U.S. Cl. 424/172.1; 530/389.1; 530/345; 514/13.3; 536/24.5; 514/44 A

(57) ABSTRACT

The invention concerns a method for inhibiting angiogenesis or for treating or preventing cancer or cancer metastasis in an individual. The method is based on the administration of an agent capable of counteracting the influence of or for down-regulating the expression of the vascular adhesion protein 1 (VAP-I). The inhibition of the catalytic activity of VAP-I is especially desirable for inhibiting angiogenesis and for treating or preventing of cancer or cancer metastasis.

### FIELD OF THE INVENTION

This invention concerns a method for inhibiting angiogenesis or for treating or preventing cancer or cancer metastasis in an individual. The method is based on the administration of an agent capable of counteracting the influence of or for down-regulating the expression of vascular adhesion protein 1 (VAP-1). The inhibition of the catalytic activity of VAP-1 is especially desirable for inhibiting angiogenesis and for treating or preventing of cancer or cancer metastasis.

### BACKGROUND OF THE INVENTION

The publications and other materials used herein to illuminate the background of the invention, and in particular, cases to provide additional details respecting the practice, are incorporated by reference.

VAP-1 is a human endothelial cell adhesion molecule that has several unique properties that distinguish it from the other inflammation-related adhesion molecules. It has a unique and restricted expression pattern and mediates lymphocyte binding to vascular endothelium (Salmi, M., and Jalkanen, S., Science 257:1407-1409 (1992)). Inflammation induces the upregulation of VAP-1 to the surface of vascular endothelial cells mediating leukocyte entry to skin, gut and inflamed synovium (Salmi, M., and Jalkanen, S., Science 257:1407-1409 (1992); Salmi, M, et al., J. Exp. Med. 178:2255-2260 (1993); Arvilommi, A., et al., Eur. J. Immunol. 26:825-833 (1996); Salmi, M., et al., J. Clin. Invest. 99:2165-2172 (1997): (Salmi. M., and Jalkanen, S., J. Exp. Med. 183:569-579 (1996); J. Exp. Med. 186:589-600 (1997)). One of the most interesting features of VAP-1 is a catalytic extracellular domain which contains a monoamine oxidase activity (Smith, D. J., et al., J. Exp. Med. 188:17-27 (1998)).

The cloning and sequencing of the human VAP-1 cDNA revealed that it encodes a transmembrane protein with homology to a class of enzymes called the copper-containing amine oxidases (E.C. 1.4.3.6). Enzyme assays have shown that VAP-1 possesses a monoamine oxidase (MAO) activity which is present in the extracellular domain of the protein (Smith, D. J., et al., J. Exp. Med. 188:17-27 (1998)). Thus, VAP-1 is an ecto-enzyme. Analysis of the VAP-1 MAO activity showed that VAP-1 belongs to the class of membrane-bound MAO's termed semicarbazide-sensitive amine oxidases (SSAO). These are distinguished from the widely distributed mitochondrial MAO-A and B flavoproteins by amino acid sequence, cofactor, substrate specificity and sensitivity to certain inhibitors. However, certain substrates and inhibitors are common to both SSAO and MAO activities.

Leukocyte trafficking from blood to tissues is not only a prerequisite for generating normal immune responses against microbes but it is also needed for immunosurveillance against malignantly transformed cells. Normally leukocytes leave the blood using a multistep extravasation cascade involving many activation and adhesion molecules both on the leukocyte and on the endothelial lining VAP-1 is one endothelial molecule that supports rolling, firm adhesion, and transmigration of various subsets of leukocytes into sites of inflammation (Salmi, M., and S. Jalkanen. 2005. Nat. Rev. Immunol. 5:760-771). VAP-1 belongs to semicarbazide sensitive amine oxidases, which are enzymes that catalyze oxidative deamination of amines into corresponding aldehydes in a reaction that also produces hydrogen peroxide and ammonium. The adhesive role of VAP-1 in leukocyte trafficking can be inhibited using function-blocking mAbs or enzyme inhibitors in multiple in vitro and in vivo inflammation models (Salmi, M., and S. Jalkanen. 2005. Nat. Rev. Immunol. 5:760-771). The anti-VAP-1 antibodies do not inhibit the enzymatic activity of VAP-1, and the enzyme inhibitors do not alter the mAb-defined surface epitopes of VAP-1 (Koskinen, K., P. J. Vainio, D. J. Smith, M. Pihlavisto, S. Yla-Herttuala, S. Jalkanen, and M. Salmi. 2004. Blood 103:3388-3395; Bonder, C., M. G. Swain, L. D. Zbytnuik, M. U. Norman, J. Yamanouchi, P. Santamaria, M. Ajuebor, M. Salmi, S. Jalkanen, and P. Kubes. 2005. Immunity 23:153-163). It is thus thought that VAP-1 is involved in leukocyte extravasation by serving as a traditional adhesion molecule (mAb-defined epitopes) and as an enzyme (by reacting with surface displayed amines of leukocytes) (Salmi, M., and S. Jalkanen. 2005. Nat. Rev. Immunol. 5:760-771).

Various strategies for inhibiting VAP-1 activity have been disclosed. For example, WO 93/25582 discloses a monoclonal antibody specifically binding to VAP-1. WO 2003/093319 describes a humanized anti-VAP-1 monoclonal antibody.

Alternatively, VAP-1 can be counteracted by using small molecules as inhibitors. The patent publications WO 2002/020290, WO 2002/002541, WO 2003/006003 and WO 2005/080319 disclose certain hydrazino compounds useful as specific VAP-1 SSAO inhibitors that modulate VAP-1 activity. These compounds are described as useful for the treatment of acute and chronic inflammatory conditions or diseases as well as diseases related to carbohydrate metabolism, aberrations in adipocyte differentiation or function and smooth muscle cell function, and various vascular diseases.

WO 2006/128951 discloses the conjugation of a small molecule inhibitor to a peptide capable of binding to VAP-1, where the peptide has a sequence of 7 to 9 amino acids, and where at least one lysine residue is located in the mid-portion of the sequence.

WO 2006/134203 discloses down-regulation of the expression of VAP-1 by use of a small interfering RNA (siRNA), which is a duplex comprising an antisense sequence of about 21 nucleotides, where the antisense is complementary to a region of the VAP-1 mRNA, and a sense sequence that is complementary to a sequence of about 19 nucleotides of the antisense.

Prior art does not disclose any relationship between SSAO-activity and formation of blood vessels. Especially, no relationship between decreased VAP-1 activity and decreased tumour growth has been published. Garpenstrand et al., Medical Oncology, vol. 2, no. 3, 241-250, 2004, describes a study aiming at clarifying the correlation between SSAO-activity and VEGF (vascular endothelial growth factor) in non-small-cell lung cancer patients. A correlation in serum between SSAO-activity and VEGF was reported. They found that an increased SSAO activity correlated with decreased VEGF concentrations. Also, serum SSAO activity did not correlate to the survival of the lung cancer patients.

Unexpectedly, the inventors of the present invention found a positive relationship between blood vessels growth and VAP-1 activity. Accordingly, they also found that a decreased VAP-1 activity inhibited tumour growth.

### SUMMARY OF THE INVENTION

The present invention is based on a study carried out by the inventors and presented in detail below. They found that the growth of melanoma and lymphoma was slower in VAP-1 deficient mice when compared to wild-type controls. They observed that the formation of new blood vessels nourishing the tumor is retarded in the absence of VAP-1. Notably, when the enzymatic activity of VAP-1 was neutralized using small molecule enzyme inhibitors, the neoangiogenesis and growth of melanoma and lymphoma were also defective. These data show that VAP-1 plays a novel role in controlling the formation of blood vessels. Furthermore, inhibition of VAP-1 can be envisaged to block tumor growth.

Thus, according to one aspect, the invention concerns the use of an agent capable of counteracting the influence of or for down-regulating the expression of vascular adhesion protein 1 (VAP-1) in an individual for the preparation of a pharmaceutical composition for inhibiting angiogenesis or for treatment or prevention of a disease benefiting from suppression of the growth of blood vessels.

According to another aspect, the invention concerns the use of an agent capable of counteracting the influence of or for down-regulating the expression of VAP-1 in an individual for the preparation of a pharmaceutical composition for use in treatment or prevention of cancer or cancer metastasis.

According to a third aspect, the invention concerns a method for inhibiting angiogenesis or for treatment or prevention of a disease benefiting from suppression of the growth of blood vessels in an individual, wherein an effective amount of an agent capable of counteracting the influence of or for down-regulating the expression of VAP-1 is administered to said individual.

According to a fourth aspect, the invention concerns a method for treatment or prevention of a cancer or cancer metastasis in an individual, wherein an effective amount of an agent capable of counteracting the influence of or for down-regulating the expression of VAP-1 is administered to said individual.

According to a fifth aspect, the invention concerns an agent capable of counteracting the influence of or for down-regulating the expression of VAP-1 in an individual for inhibiting angiogenesis or for treatment or prevention of a disease benefiting from suppression of the growth of blood vessels.

According to a sixth aspect, the invention concerns an agent capable of counteracting the influence of or for down-regulating the expression of VAP-1 in an individual for use in treatment or prevention of cancer or cancer metastasis.

### BRIEF DESCRIPTION OF THE DRAWINGS

FIG. 1. Impaired growth of melanoma in VAP-1 deficient mice. A) Luciferase expressing B16 melanoma cells were injected into flanks of VAP-1 deficient and wt mice. The growth of tumor was followed for 10 d using bioluminescence imaging (n=15 wt and 14 VAP-1 −/− mice). B) The diameters of the tumor were measured using an electronic caliber and an estimate of the tumor volume was counted (n=12 wt and 11 VAP-1 −/− mice). The results are shown as mean±SEM, *, p<0.05.

FIG. 2. Inhibition of VAP-1 by mAbs or VAP-1 inhibitors retards the growth of melanoma (A). Wt mice were treated daily with the negative control or anti-VAP-1 mAb, and the growth of luciferase expressing melanoma cells was followed using bioluminescence (n=15 wt and 14 VAP-1 −/− mice). Wt mice were treated daily with the VAP-1 inhibitor SZE5302 (B), and the diameter of the tumor was measured (n=12 wt and 11 VAP-1 −/− mice). The results are shown as mean±SEM, **, p<0.01.

FIG. 3. A VAP-1 inhibitor prevents the neoangiogenesis in melanoma tumors. The tumors were resected from the mAb and VAP-1 inhibitor-treated groups at the end of the experiment (d10). The number of CD31-positive vessels inside the tumor was determined using immunohistochemistry and microscopic counting. The results are shown as mean±SEM (n=5 mice/group), **, p<0.01. HPF=high power field (400× magnification).

FIG. 4. Impaired growth of neovessels into tumor cell-containing Matrigel plugs in the absence of VAP-1. Matrigel plugs containing melanoma cells were injected into wt and VAP-1 deficient mice. After 10 d, the plugs were excised and the number of vessels infiltrating into the Matrigel was determined microscopically from HE-stained sections. The results are shown as mean±SEM (n=5 mice/group), *, p<0.05. HPF=high power field (400× magnification).

FIG. 5. Defective migration of myeloid-derived suppressor cells (MDSC), angiogenesis and growth of lymphoma in VAP-1 deficient mice (a, b). EL-4 lymphoma cells were injected subcutaneously into (a) wt and VAP-1 deficient mice and (b) control and SZE5302 treated mice, and the growth of the lymphomas (volume) was measured kinetically. VAP-1 activity in control and SZE5302 treated mice was measured using enzyme assay (c) Immunohistochemical stainings of (d) VAP-1 and (e) CD31 expression in tumor vessels in EL-4 lymphoma grown in wt and VAP-1 −/− mice. Some positive vessels are pointed out by arrows. Lymphoma cells also express low levels of CD31. Bar 50 μm. The numbers of (f) CD31 positive vessels and (g) CD11b positive cells was determined using immunohistochemistry. Data (mean±SEM) are from 5-6 mice per group. *, p<0.05, ** p<0.01.

FIG. 6. VAP-1 selectively supports recruitment of pro-angiogenic myeloid-derived suppressor cells in melanoma tumors. CD11b-positive cells were quantified by microscopic counting in wt and VAP-1 deficient mice and in wt treated with vehicle or SZE5302 inhibitor. Data (mean±SEM) are from 9 mice per group, ** p<0.01.

### DETAILED DESCRIPTION OF THE INVENTION

The term “treatment” or “treating” shall be understood to include complete curing of a disease or disorder, as well as amelioration or alleviation of said disease or disorder.

The term “prevention” shall be understood to include complete prevention, prophylaxis, as well as lowering the individual's risk of falling ill with said disease or disorder.

The term “individual” refers to a human or animal subject.

The term “effective amount” is meant to include any amount of an agent according to the present invention that is sufficient to bring about a desired therapeutical result, especially upon administration to an animal or human subject.

The term “inhibiting” or “inhibition” shall be understood to include not only complete inhibition but also any grade of suppression.

### Preferred Agents

The term “an agent capable of counteracting the influence of vascular adhesion protein 1” shall be understood to include antibodies blocking the protein as well as any inhibitors, particularly small molecule inhibitors useful to inhibit the enzyme activity.

The term “an agent capable of down-regulating the expression of vascular adhesion protein 1” shall be understood to include antisense oligonucleotides, small interfering RNAs (siRNA) as well as ribozymes, or vectors being capable of expressing them, or essential parts thereof, in vivo.

The term “antibody” shall be understood to include monoclonal and polyclonal antibodies and any fragments thereof. Also genetically engineered antibodies and fragments are included. For use in human individuals, humanized or chimeric antibodies are preferred. Such antibodies are described for example in WO 2003/093319.

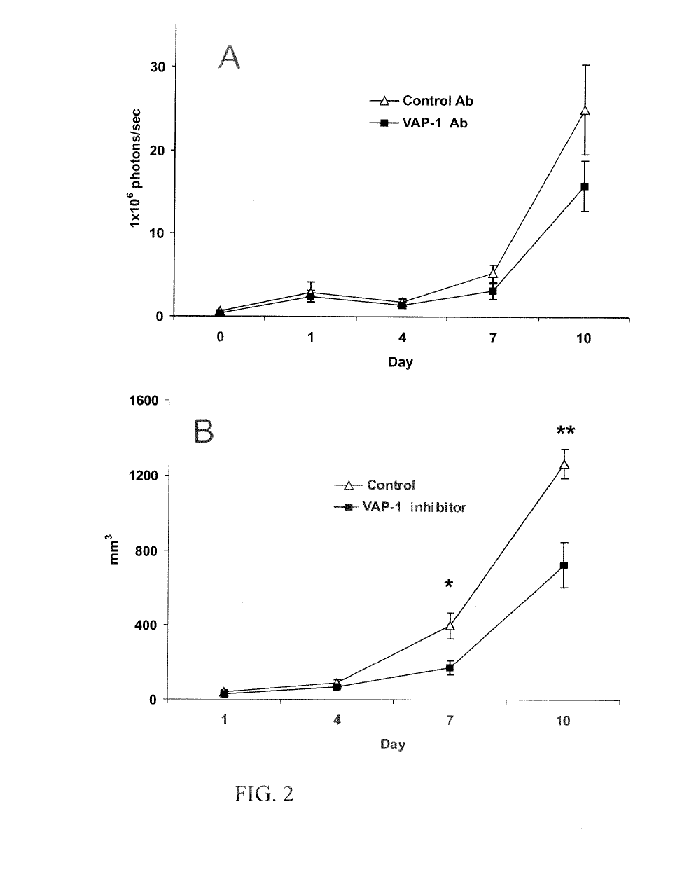

The preferred method of inhibiting VAP-1 for the treatment of cancer and preventing angiogenesis is to block its enzymatic activity. Based on the experimental data this is more effective than blocking the adhesive function of VAP-1 by antibodies in the inhibition of tumor growth (FIG. 2). It can be hypothesized that the production of aldehydes, hydrogen peroxide and ammonium, or any one of them, is normally needed for neoangiogenesis and/or other growth promoting effects in tumors. Therefore, the inhibition of their production by inhibiting the enzymatic activity of VAP-1 would impair neoangiogenesis and/or other aspects of tumor growth.

Preferable inhibitors are small molecule inhibitors. Small molecule VAP-1 inhibitors are, for example, those disclosed in the art; see WO 2002/020290, WO 2002/002541, WO 2003/006003 and WO 2005/080319.

A preferred small molecule inhibitor is the compound coded SZE5302 ((1S,2S)-(1-methylhydrazino)-1-indanol), which also is known under the code BTT2052. The compound is described for example in Fumiko Marttila-Ichihara et al., Arthritis & Rheumatism, vol. 54, no. 9 Sep. 2006, pp. 2852-2862. Another preferred small molecule is LJP1586 ([Z-3-fluoro-2-(4-methoxybenzyl)allylamine hydrochloride]. These small molecule inhibitors are mentioned as non-restrictive examples only.

The inhibitors, such as small molecule inhibitors can be administered as such, preferably mixed with a suitable pharmaceutically acceptable carrier, or in conjugation with a peptide, which is able to bind to the VAP-1 enzyme. Such VAP-1 binding peptides are disclosed in WO 2006/128951 A1. The peptide comprises an amino acid sequence of at least 7 amino acids, preferably 7 to 9 amino acids, where the sequence comprising a mid-part having at least one lysine residue in the mid-part of the sequence, and terminal parts, each terminal part preferably comprising at least two consecutive glycine residues. Particularly preferred examples of such peptides are listed in claim 13 of WO 2006/128951 A1. The linking of the peptide to the small molecule inhibitor can be carried out as described on page 8, lines 16-26, of the publication.

Other useful peptides capable of binding to VAP-1 are described in the art, for example in Elina Kivi, Identification of ligands for endothelial receptor VAP-1 by using phage display. Master thesis, January 2007, Department of Biology, University of Turku. Useful VAP-1 binding peptides are, for example, the sequences CVKWRGVVVC (SEQ ID NO. 1) or CWSFRNRVLC (SEQ ID NO. 2), or their homologues having at least 4 amino acids in common with these sequences. A special group of peptides are those derived from proteins belonging to the Siglec or ADAM group or to the CD58 glycoproteins. As specific examples were mentioned CARLSLSWRGLTLCPS (SEQ ID NO. 3), CATLSWVLQNRVLSSC (SEQ ID NO. 4) and CLENFSKWRGSVLSRRC (SEQ ID NO. 5).

Preferably, the agent capable of down-regulating the expression of a SSAO, especially VAP-1, is a small interfering RNAs (siRNA) or an expression vector comprising nucleic acid encoding the siRNA duplex or the antisense strand of the duplex in a manner which allows expression of the siRNA duplex or antisense strand within a mammalian cell. Such VAP-1 siRNA duplexes are described in WO 2006/134203 A1. Preferably, the siRNA is a duplex comprising an antisense sequence of about 21 nucleotides, where the antisense is complementary to a region of the VAP-1 mRNA, and a sense sequence that is complementary to a sequence of about 19 nucleotides of the antisense. Particularly preferred examples of VAP-1 siRNA-duplexes are shown in FIG. 5 of WO 2006/134203 A1.

The principle of siRNA is extensively presented in literature. As examples can be mentioned the US patent publications 2003/0143732, 2003/0148507, 2003/0175950, 2003/0190635, 2004/0019001, 2005/0008617 and 2005/0043266. An siRNA duplex molecule comprises an antisense region and a sense strand wherein said antisense strand comprises sequence complementary to a target region in an mRNA sequence encoding a certain protein, and the sense strand comprises sequence complementary to the said antisense strand. Thus, the siRNA duplex molecule is assembled from two nucleic acid fragments wherein one fragment comprises the antisense strand and the second fragment comprises the sense strand of said siRNA molecule. The sense strand and antisense strand can be covalently connected via a linker molecule, which can be a polynucleotide linker or a non-nucleotide linker. The length of the antisense and sense strands are typically about 19 to 21 nucleotides each. Typically, the antisense strand and the sense strand both comprise a 3′-terminal overhang of a few, typically 2 nucleotides. The 5′-terminal of the antisense is typically a phosphate group (P). The siRNA duplexes having terminal phosphate groups (P) are easier to administrate into the cell than a single stranded antisense. In the cell, an active siRNA antisense strand is formed and it recognizes a target region of the target mRNA. This in turn leads to cleaving of the target RNA by the RISC endonuclease complex (RISC=RNA-induced silencing complex) and also in the synthesis of additional RNA by RNA dependent RNA polymerase (RdRP), which can activate DICER and result in additional siRNA duplex molecules, thereby amplifying the response.

The term “complementary” means that the nucleotide sequence can form hydrogen bonds with the target RNA sequence by Watson-Crick or other base-pair interactions. The term shall be understood to cover also sequences which are not 100% complementary. It is believed that also lower complementarity might work. However, 100% complementarity is preferred.

The siRNA shall, when used as a pharmaceutical, be introduced in a target cell. The delivery can be accomplished in two principally different ways: 1) exogenous delivery of the oligonucleotide or 2) endogenous transcription of a DNA sequence encoding the oligonucleotide, where the DNA sequence is located in a vector.

Normal, unmodified RNA has low stability under physiological conditions because of its degradation by ribonuclease enzymes present in the living cell. If the oligonucleotide shall be administered exogenously, it is highly desirable to modify the molecule according to known methods so as to enhance its stability against chemical and enzymatic degradation.

Modifications of nucleotides to be administered exogenously in vivo are extensively described in the art. Principally, any part of the nucleotide, i.e the ribose sugar, the base and/or internucleotidic phosphodiester strands can be modified. It should be stressed that the modifications mentioned above are only non-limiting examples.

Although the VAP-1 siRNA duplexes and vectors described in WO 2006/134203, it is stressed that also other useful siRNA:s based on other target regions at the target RNA can be used. A useful target region can easily be identified by using any of the numerous academic or commercially affiliated algorithms that have been developed to assist scientists to locate utilizable siRNA sequences. As examples of such software systems can be mentioned siDirect (http://design.RNAi.jp/) (Nucleic Acids Res. 2004 Jul. 1; 32: W124-9); TROD (T7 RNAi Oligo Designer (http://www.cellbio.unige.ch/RNAi.html; Nucleic Acids Res. 2004 Jul. 1; 32: W121-3); DEQOR (http://cluster-1.mpi-cbg.de/Deqor/deqor.html; Nucleic Acids Res. 2004 Jul. 1; 32: W113-20) or programs available at http://www.genscript.com; http://www.genscript.com/rnai.html#design or http://www.genscript.com/sirna_ca.html#design; Bioinformatics 2004 Jul. 22; 20(11) 1818-20. An essential criterion of the tools is to achieve siRNA:s with maximum target-specificity for mammalian RNA interference where off-target gene silencing is avoided. The usefulness of any sequence identified by such algorithms should thereafter be verified by experiments.

The use of VAP-1 inhibition together with other forms of angiostatic drugs (such as VEGF blocking antibodies) may provide additional or synergistic benefits to the patient needing such therapy.

### Diseases Responding to the Treatment

This method according to this invention is useful for treatment or prevention of any disease benefiting from suppression of the growth of blood vessels in an individual. As non-restricting examples of such diseases can be mentioned arthritis, retinopathy, age related macular degeneration, atherosclerosis, and all forms of cancers. Thus, any benign or malignant tumour or metastasis of malignant tumor, such as skin cancer and colon cancer can be treated. Also leukemias, lymphomas and multiple myelomas can be treated. Particularly, melanomas and lymphomas have been shown to respond very well to the treatment.

Based on the experiments carried out we believe that the method according to this invention is useful in the treatment or prevention of all kinds of sarcomas, for example fibrosarcoma, liposarcoma, chondrosarcoma, osteosarcoma, angiosarcoma, lymphangisarcoma, leiomyosarcoma, and rhabdomyosarcoma, mesothelioma, meningoma, leukemias, lymphomas, as well as all kinds of carcinomas, such as squamous cell carcinomas, basal cell carcinoma, adenocarcinomas, papillary carcinomas, cystadenocarcinomas, bronchogenic carcinomas, melanomas, renal cell carcinomas, hepatocellular carcinoma, transitional cell carcinomas, choriocarcinomas, seminomas, and embryonal carcinomas.

Although we believe that the beneficial effect on cancers and cancer metastasis is mainly based on the suppression of the growth of blood vessels, its should be noted that also other mechanisms may be involved.

### Administration Routes, Formulations and Required Dose

The pharmaceutical compositions to be used in the present invention can be administered by any means that achieve their intended purpose. For example, administration can be by parenteral, subcutaneous, intravenous, intraarticular, intrathecal, intramuscular, intraperitoneal, or intradermal injections, or by transdermal, buccal, ocular routes or via inhalation. Alternatively, administration can be by the oral route. Particularly preferred for small molecule inhibitors may be oral administration. In addition to the pharmacologically active compounds, the pharmaceutical preparations of the compounds preferably contain suitable pharmaceutically acceptable carriers comprising excipients and auxiliaries that facilitate processing of the active compounds into preparations that can be used pharmaceutically.

The siRNA duplex for use in this invention can be administered to the individual by various methods. According to one method, the siRNA may be administered exogenously as such, or in the form of a pharmaceutical composition admixed with a suitable carrier which may be, for example, a liposome, cholesterol, lithocholic acid, lauric acid, a cationic lipid, polyethylenimine (PEI) or its conjugates with polyethylene glycol (PEG) derivatives. However, also other carriers can be used. The siRNA can be administered systemically or locally. As suitable routes of administration can be mentioned intravenous, intramuscular, subcutaneous injection, inhalation, oral, topical, ocular, sublingual, nasal, rectal, intraperitoneal delivery and transdermal delivery systems. The composition containing the siRNA can, instead of using direct injection, also be administered by use of, for example, a catheter, infusion pump or stent.

Another method to achieve high concentrations of the siRNA in cells is to incorporate the siRNA-encoding sequence into an expression vector and to administer such a vector to the individual. In this application, the expression vector could be construed so that either the siRNA duplex or only the antisense strand thereof is expressed, e.g. in the form of short hairpin RNAs. The expression vector can be a DNA sequence, such as a DNA plasmid capable of eukaryotic expression, or a viral vector. Such a viral vector is preferably based on an adenovirus, an alphavirus, an adeno-associated virus or a retrovirus. Preferably, the vector is delivered to the patient in similar manner as the siRNA described above. The delivery of the expression vector can be systemic, such as intravenous, intramuscular or intraperitoneal administration, or local delivery to target tissue or to cells explanted from the patient, followed by reintroduction into the patient.

Since intravenous administration of siRNA preferentially targets liver vasculature (Lewis D L and Wolff J A, Methods Enzymol. 2005; 392:336-50; Soutschek J et al., Nature. 2004 Nov. 11; 432(7014):173-8; and Song E et al., Nat. Med. 2003 March; 9(3):347-51), diseases of liver are especially suitable targets for intervention. Especially siRNA:s embedded in liposoms have been reported to be very useful for targeting liver tissue. No toxic side-effects have been reported.

Thus, a typical dose is in the dosage range of about 0.1 microgram/kg to about 300 mg/kg, preferably between 1.0 microgram/kg to 10 mg/kg body weight. Compounds for use in the present invention may be administered in a single daily dose, or the total daily dosage may be administered in divided doses of two, three or four times daily. When siRNA is used, a typical daily dose is in the dosage range of about 1 mg/kg to about 20 mg/kg, preferably about 5 mg/kg body weight. The suitable administration frequence is believed to be 1 to 2 doses daily. When the RNAi is delivered by an expression vector, a single dose (or a single doses repeated at certain intervals, eg. once in week) is believed to be enough.

The invention will be illuminated by the following non-restrictive Experimental Section.

### EXPERIMENTAL SECTION

Blocking of VAP-1 function effectively attenuates inflammatory reactions in vivo, and therapeutics aiming at inhibition of VAP-1 are actively been developed to treat inflammatory disorders. However, it is not known whether VAP-1 is involved in anti-tumor immunity. It has earlier been shown in vitro that NK cells and tumor infiltrating lymphocytes can use VAP-1 to bind to vessels in frozen section binding assays (Irjala, H., M. Salmi, K. Alanen, R. Grév nman, and S. Jalkanen. 2001. J. Immunol. 166:6937-6943). However, nothing is known about the role of VAP-1 in tumor growth in vivo. Here we studied the role of VAP-1 in melanomas and lymphomas using VAP-1 deficient mice, anti-VAP-1 mAbs and VAP-1 inhibitors.

We found that VAP-1 activity is needed for normal neoangiogenesis and tumor growth. These data reveal a previously unknown function for VAP-1 in regulation of angiogenesis and indicate that blocking of VAP-1 can be used to attenuate tumor growth.

### Materials and Methods

### Tumor Models

In the melanoma model, age- and sex-matched VAP-1 deficient and wt C57/B16 mice were used (Stolen, C. M., F. Marttila-Ichihara, K. Koskinen, G. G. Yegutkin, R. Turja, P. Bono, M. Skurnik, A. Hanninen, S. Jalkanen, and M. Salmi. 2005. Immunity 22:105-115). Luciferase-containing B16 melanoma cells were purchased from Xenogen. Tumor cells (4×10⁵ in 200 μl) were injected subcutaneously into the flanks of isoflurane anesthesized mice. Immediately thereafter, a substrate for the luciferase (D-luciferin sodium salt, Synchem, Kassel, Germany; 150 mg/kg i.p.) was injected, and 10 min later the light generated was recorded using bioluminescence imaging (IVIS 50 workstation, Xenogen). The signal intensities were quantified as the photon counts using the Living Image software (Xenogen). The photon counts have been shown to be a reliable indicator of the tumor cell number (Xenogen and e.g. Minn, A. J., Y. Kang, I. Serganova, G. P. Gupta, D. D. Giri, M. Doubrovin, V. Ponomarev, W. L. Gerald, R. Blasberg, and J. Massague. 2005. J. Clin. Invest. 115:44-55). The growth of tumors was then followed for 10 d, and at the indicated time points, the animals were re-subjected to the bioluminescence imaging. The diameter of the tumor was also measured using an electronic caliber (Mitutoyo, Japan). The volume of the tumor was approximated using the formula: 0.5×(longest diameter)×(shortest diameter). In the lymphoma model, EL-4 cells were injected into the abdominal area (10×10⁶ cells/mouse in 200 μl of RPMI1640. tumor growth was followed using the electronic caliber.

### Anti-VAP-1 Antibody and VAP-1 Inhibitor Treatments

The tumor model was established as above. The animals were treated every second day with anti-VAP-1 mAb (7-106+7-88, 100 μg each/mouse) or with a negative control mAb HB151 (200 μg/mouse, ip) (Merinen, M., H. Irjala, M. Salmi, I. Jaakkola, A. Hanninen, and S. Jalkanen. 2005. e. Am. J. Pathol. 166:793-800). The antibodies were produced in serum and BSA-free medium, concentrated, and dialyzed against PBS. Another group of mice were treated with the VAP-1 inhibitor SZE 5302 (50 mg/kg, ip, daily) or with the vehicle (saline) (Koskinen, K., P. J. Vainio, D. J. Smith, M. Pihlavisto, S. Yla-Herttuala, S. Jalkanen, and M. Salmi. 2004. Blood 103:3388-3395). The growth of the tumor was then followed using bioluminescence imaging or volume measurements as described above.

### Immunohistochemistry

At the end of the experiment, tumors were excised from the mice and processed for immunohistochemical staining. Anti-mouse CD31 mAb (MEC13.3, IHC formula, 1:300 dilution; Pharmingen) and a negative control mAb (9B5) were used for indirect immunoperoxidase stainings. The sections were counterstained with hematoxylin-eosin, and mounted in Depex. In certain cases, formalin-fixed, paraffin embedded sections were stained with HE. The CD31-positive vessels or vessels in HE-stained sections were enumerated by counting the number of vessels in the whole tumor area using a 400× magnification. Sections were also stained with CD11b and Gr-1 antibodies. FACS results showed that >70% of CD11b positive cells in tumors co-expressed Gr-1 (data not shown), which defines them as myeloid-derived suppressor cells.

### Matrigel Plugs

Melanoma cells (4×10⁵ in 200 μl) were mixed with Marigel (300 μl; a basement membrane extract, Becton-Dickinson) and injected to the flanks of recipient mice according to the manufacturer's instructions. After 10 days the plugs were excised, and the number of new vessels in the plugs was quantified using CD31 stainings as described above.

### Statistical Analyses

Unpaired, two-tailed Student's t-test was used for statistical analyses. P-values <0.05 were considered significant.

### Results

Retarded growth of melanoma in VAP-1 deficient mice

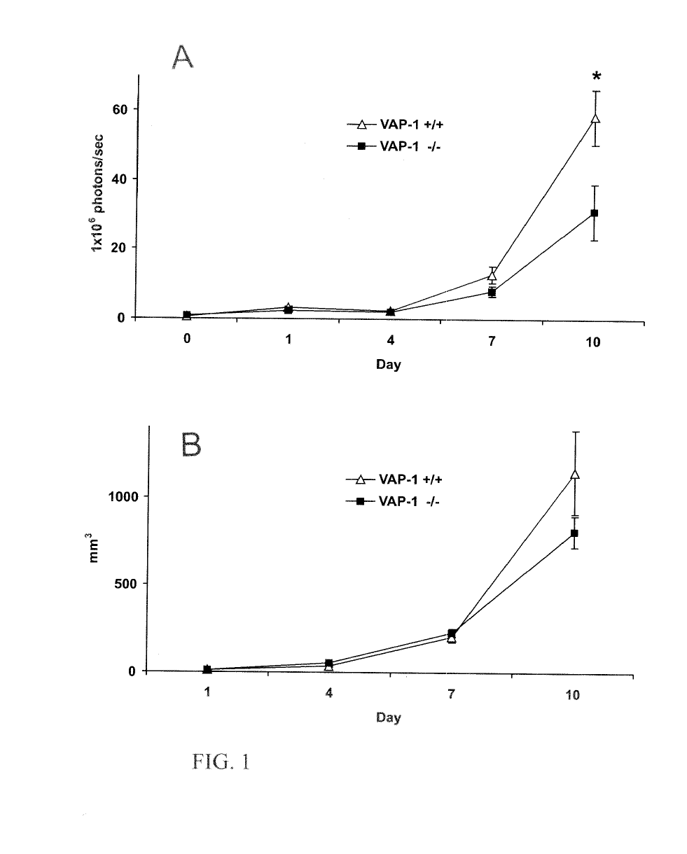

To study the role of VAP-1 in tumor growth we injected luciferase-tagged B16 melanoma cells subcutaneously to wild type and VAP-1 KO mice. When the growth of melanoma cells was kinetically followed using bioluminescence imaging we found that tumors were formed in both genotypes. After an initial period of similar growth, the tumors started to grow slower in VAP-1 deficient mice at day 7 (FIG. 1A). At day 10, when the mice had to be euthanized due to ethical reasons, tumors were significantly smaller in VAP-1 deficient mice when compared to wild-type controls. When an estimate of the tumor volume was obtained by measuring the tumor diameters, similar attenuation of tumor growth in VAP-1 deficient mice was evident (FIG. 1B). These data thus show that the growth of melanomas is retarded in the absence of VAP-1.

### The Enzymatic Activity of VAP-1 is Important for Tumor Growth

The function of VAP-1 can be therapeutically inhibited by either using anti-VAP-1 mAbs or VAP-1 enzyme inhibitors (Salmi, M., and S. Jalkanen. 2005. Nat. Rev. Immunol. 5:760-771). We therefore injected melanoma cells into mice and treated the animals with function-blocking anti-VAP-1 mAb or a isotype-matched control antibody (daily i.p. injections). When the function of VAP-1 was blocked with the antibodies, the tumor cell number tended to grow slower, although the difference did not reach statistical significance (FIG. 2A).

When the mice were treated with a selective VAP-1 inhibitor SZE5302, a significant decrease in tumor growth was observed (FIG. 2B). The inhibitor interfered with the luciferase-based assay, but physical measurement of the tumor volume showed a significant reduction in SZE-treated mice already on day 7. At day 10 the melanomas were clearly smaller in the absence of VAP-1 activity. Thus, the catalytical activity of VAP-1 supports the growth of melanoma tumors.

### VAP-1 Supports Neoangiogenesis in Tumors

VAP-1 is expressed in the endothelium, pericytes and smooth muscle cells in vessels (Salmi, M, et al., J. Exp. Med. 178:2255-2260 (1993); Salmi, M., and Jalkanen, S., Science 257:1407-1409 (1992)). We therefore asked if the neutralization of VAP-1 would alter the vasculature in tumors. To that end we immunostained tumors grown in wt mice treated or not with the anti-VAP-1 antibodies or enzyme inhibitor.

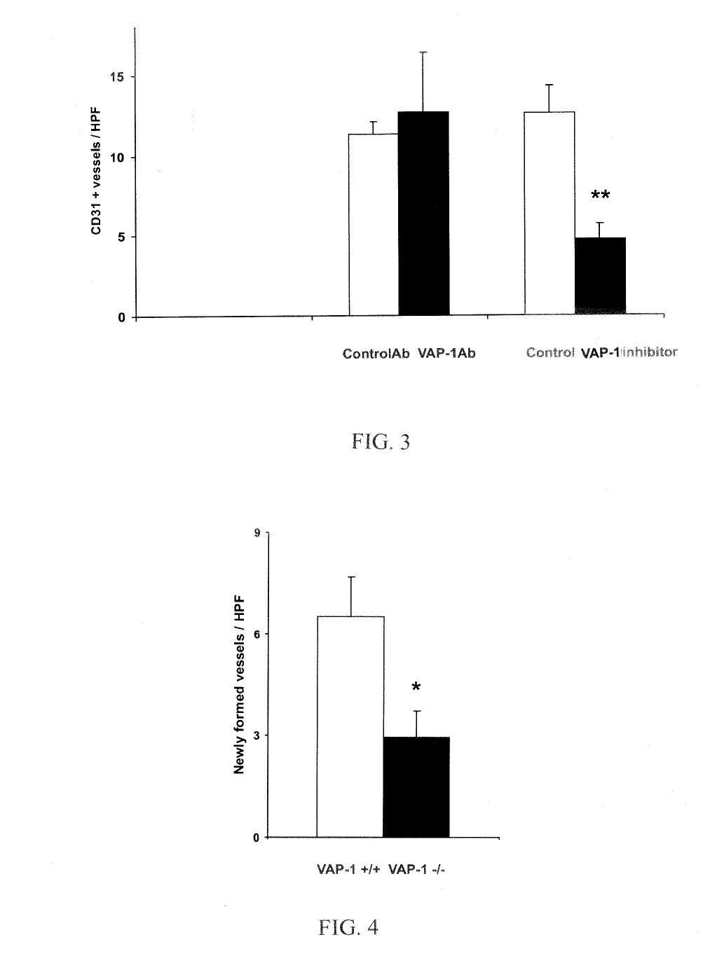

We found that the anti-VAP-1 antibody treatment did not affect the number of new vessels in the tumor. In contrast, there were significantly fewer CD31+ positive vessels in the tumors grown in mice treated with the VAP-1 inhibitor than in control mice (FIG. 3). These data show that the enzymatic activity of VAP-1 is involved in the regulation of formation of new vessels in tumors.

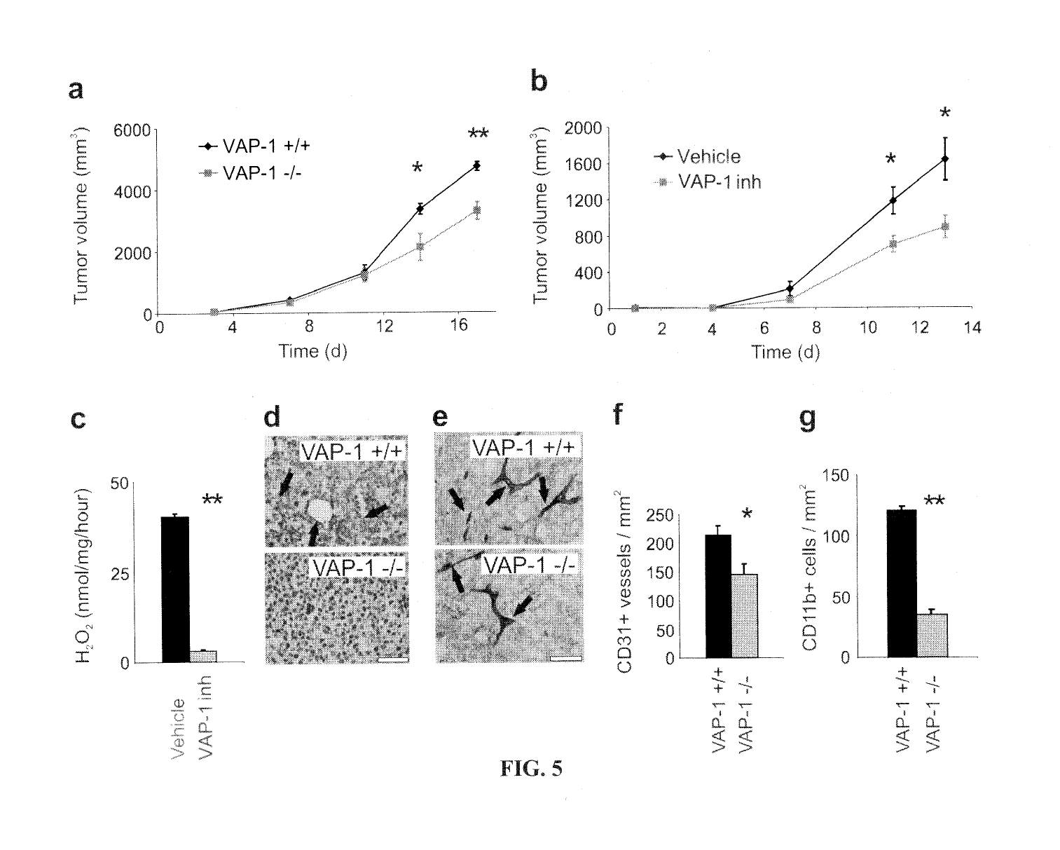

The growth of new vessels into Matrigel plugs containing melanoma cells was measured in wt and VAP-1 KO mice. It was evident that the growth of new vessels was also impaired in this model of neoangiogenesis in the absence of VAP-1 (FIG. 4).

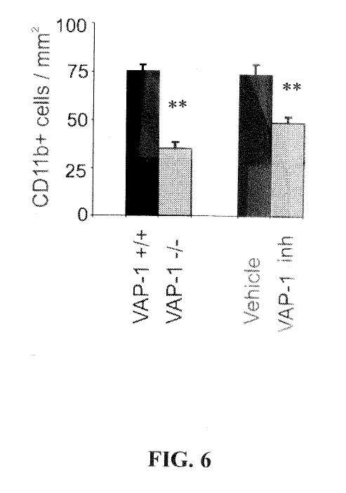

FIG. 6 shows that the number of MDSC was also strongly reduced in the melanomas in the absence of VAP-1 and in wt mice treated with the VAP-1 inhibitor SZE5302. The data show that VAP-1 supports angiogenesis by regulating migration of MDSC in melanoma tumors.

### VAP-1 is Needed for Local Growth of Lymphoma

We also asked whether VAP-1 is needed for normal expansion of different tumor cell types. We observed that after subcutaneous injections of IL-4 cells significantly smaller tumors developed in VAP-1 deficient mice when compared to wt hosts at days 14 and 17 (FIG. 5a). Treatment of wt mice with the VAP-1 inhibitor SZE5302 also strongly inhibited tumor growth (FIG. 5b, c). Neovessels within the lymphoma expressed VAP-1 in wt (FIG. 5d). Notably, the number of CD31 positive vessels was reduced in within the lymphoma in the absence of VAP-1 (FIG. 5e, f). Most importantly, the number of MDSC was also strongly reduced (71±3%, n=5 mice/group, p<0.0001) in the lymphomas in the absence of VAP-1 (FIG. 5g). These data suggests that VAP-1 supports angiogenesis by regulating migration of MDSC in several tumor types.

### CONCLUSION

These data show that VAP-1 is needed for the normal growth of melanomas and lymphomas. When the function of VAP-1 is inhibited, either through genetic deletion or through the use of anti-VAP-1 antibodies or VAP-1 inhibitors, the growth of melanoma or lymphoma cells is impaired in in vivo models. Moreover, at least in VAP-1 deficient mice and in wt animals treated with the VAP-1 inhibitor the tumor-driven neoangiogenesis was impaired. These data suggest that therapeutic inhibition or down-regulation of VAP-1 can be used to inhibit angiogenesis, particularly the vascularization of tumors and, consequently, tumor growth.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

1. Use of an agent capable of counteracting the influence of or for down-regulating the expression of vascular adhesion protein 1 (VAP-1) in an individual for the preparation of a pharmaceutical composition for inhibiting angiogenesis or for treatment or prevention of a disease benefiting from suppression of the growth of blood vessels.

2. Use of an agent capable of counteracting the influence of or for down-regulating the expression of vascular adhesion protein 1 (VAP-1) in an individual for the preparation of a pharmaceutical composition for use in treatment or prevention of cancer or cancer metastasis.

3. The use according to claim 1 wherein the disease is cancer or cancer metastasis.

4. The use according to claim 2 wherein the growth of the cancer is inhibited by inhibiting the formation of new blood vessels in the cancer.

5. The use according to claim 1, wherein the agent is (a) a VAP-1 antibody, (b) a VAP-1 inhibitor especially a small molecule inhibitor of VAP-1, (c) a small molecule inhibitor of VAP-1 conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

6. The use according to claim 2, wherein the agent is (a) a VAP-1 antibody, (b) a VAP-1 inhibitor, especially a small molecule inhibitor of VAP-1, (c) a small molecule inhibitor of VAP-1 conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

7. The use according to claim 3, wherein the agent comprises (a) a VAP-1 antibody, (b) a VAP-1 inhibitor especially a small molecule inhibitor of VAP-1, (c) a small molecule inhibitor conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

8. The use according to claim 4, wherein the agent is (a) a VAP-1 antibody, (b) a VAP-1 inhibitor especially a small molecule inhibitor of VAP-1, (c) a small molecule inhibitor conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

9. The use according to claim 2, wherein the cancer is a melanoma or a lymphoma.

10. The use according to claim 3, wherein the cancer is a melanoma or a lymphoma.

11. A method of inhibiting angiogenesis or of treating or preventing a disease benefiting from suppression of the growth of blood vessels in an individual comprising administering a therapeutically effective amount of an agent capable of counteracting the influence of or for down-regulating the expression of vascular adhesion protein 1 (VAP-1) in an individual in need thereof.

12. A method of treating or preventing cancer or cancer metastasis in an individual comprising administering a therapeutically effective amount of an agent capable of counteracting the influence of or for down-regulating the expression of vascular adhesion protein 1 (VAP-1) in an individual in need thereof.

13. The method of claim 11, wherein the disease is cancer or cancer metastasis.

14. The method of claim 12, wherein the growth of the cancer is inhibited by inhibiting the formation of new blood vessels in the cancer.

15. The method of claim 11, wherein the agent comprises (a) a VAP-1 antibody, (b) a VAP-1 inhibitor especially a small molecule inhibitor of VAP-1, (c) a small molecule inhibitor conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

16. The method of claim 12, wherein the agent comprises (a) a VAP-1 antibody, (b) a VAP-1 inhibitor especially a small molecule inhibitor of VAP-1, (c) a small molecule inhibitor conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

17. The method of claim 13, wherein the agent comprises (a) a VAP-1 antibody, (b) a VAP-1 inhibitor especially a small molecule inhibitor of VAP-1, (c) a small molecule inhibitor conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

18. The method of claim 14, wherein the agent comprises (a) a VAP-1 antibody, (b) a VAP-1 inhibitor especially a small molecule inhibitor of VAP-1, (c) a small molecule inhibitor conjugated to a peptide capable of binding to VAP-1, (d) a small interfering RNA (siRNA) which down-regulates the expression of VAP-1, said siRNA being a duplex comprising an antisense sequence of about 21 nucleotides, said antisense being complementary to a region of the VAP-1 mRNA, and a sense sequence that is complementary to a sequence of about 19 nucleotides of said antisense, or (e) an expression vector comprising a nucleic acid encoding the siRNA duplex, or the antisense strand of said duplex, in a manner which allows expression of said siRNA duplex or antisense strand within the individual's cell.

19. The method of claim 12, wherein the cancer is a melanoma or a lymphoma.

20. The method of claim 13, wherein the cancer is a melanoma or a lymphoma.
